(19) United States
(12) Patent Application Publication
(10) Pub. No.: US 2020/0121497 A1
(43) Pub. Date: Apr. 23, 2020

(54) HEAT PATCH

(71) Applicants: TING-JENG SHR, Taichung City (TW); KAI-MIN CHUANG, Taichung City (TW)

(72) Inventors: TING-JENG SHR, Taichung City (TW); KAI-MIN CHUANG, Taichung City (TW)

(21) Appl. No.: 16/162,803

(22) Filed: Oct. 17, 2018

(51) Int. Cl. A61F 7/03; A61F 13/02

(52) U.S. Cl. CPC A61F 7/03; A61K 9/7084; A61F 13/0233

(57) ABSTRACT

A heat patch includes a dressing layer, a release layer, an adherence layer, and a heating layer. The dressing layer is combined with the release layer and the adherence layer. The heating layer is combined with the adherence layer. When the dressing layer is adhered to the user skin, the heating structure of the heating layer generates the heat which is transmitted to the dressing layer for achieving a hot compressing function.

### TECHNICAL FIELD

The present invention relates to patches, and more particularly, to a heat patch capable of self-heating.

### BACKGROUND

After over-exercising, physical labor, or a long-period of incorrect body posture, a soreness of human body muscle occurs. For relieving the soreness to maintain normal daily activities, people usually adhere sore relieving patch which includes soreness relieving medication. With the medication permeating into the skin, the muscle soreness is relieved.

Also, a soreness relieving patch is easily applied on a body area which is felt uncomfortable by the user, so as to keep relieving the soreness symptom in a certain period of time. However, the air permeability of the soreness relieving patch is poor, easily causing an itchy or red and swollen skin. Especially for a sensitive skin, a serious allergy might be induced.

Therefore, as for a sensitive skin, a hot compressing method is usually recommended to achieve the soreness relieving effect. Common hot compressing method includes hot compressing bags containing warm water, hand warmers, or heating pads with electrically heating function.

Hot compressing method achieves a soreness relieving effect. However, the hot compressing product itself contains no soreness relieving medication content, such that the compressing product achieves a limited soreness relieving effect. Also, except for the portable hand warmer, the hot compressing bags and the heating pads must be applied at a fixed location, failing to be applicable anywhere like the soreness relieving patches.

### SUMMARY

For improving the issues above, a heat patch is disclosed by the present invention. With the dressing layer adhered to the user skin for providing a self-heating effect, the heat is transmitted to the dressing layer, so as to achieve a heat compressing and soreness relieving functions.

A heat patch in accordance with an embodiment of the present invention is provided, comprising:

a dressing layer having a first contact face and a second contact face in opposite to the first contact face;

a release layer removably disposed on the second contact face of the dressing layer;

an adherence layer having a release face and an adherence face in opposite to the release face, the first contact face of the dressing layer removably disposed on the release face; and

a heating layer having one lateral side disposed on the adherence face, the heating layer further having a heating structure for producing heat.

With such configuration, when the release layer is removed from the dressing layer, the dressing layer is allowed to be directly adhered to the skin of the user, such that the heating structure of the heating layer generates the heat which is directly transmitted to the dressing layer, thus providing a hot compressing effect. Therefore, the combination of the heating layer and the dressing layer simultaneously provide the functions of hot compressing and soreness relieving.

Further, the adherence layer is disposed between the heating layer and the dressing layer, so as to increase the adherence between the heating layer and the dressing layer, effectively preventing the heating layer from being accidentally detached from the dressing layer.

In an embodiment of the present invention, the first contact face and the second contact face of the dressing layer have viscosity. Therefore, the viscosity of the dressing layer helps adhering the dressing layer upon the skin surface of the user, so as to relieve the soreness therefrom.

In an embodiment of the present invention, the second contact face of the dressing layer has a plurality of magnetic stones. With the magnetism of the magnetic stones, the soreness relieving effect is improved.

In an embodiment of the present invention, a plurality of bores are disposed on the dressing layer and spaced with each other for increasing the permeability of the present invention. Therefore, the steam and sweat, which are produced by the user skin upon the heat being transmitted thereto through the dressing layer, are allowed to permeate from the bores, preventing the discomfort from occurring due to the steam and sweat.

In an embodiment of the present invention, the second contact face of the dressing layer are concavely provided with a plurality of grooves for increasing the permeability of the present invention. Therefore, when the heat generated by the heating structure is transmitted to the user skin through the dressing layer, the steam and sweat thus produced are allowed to be discharged from the grooves, preventing the discomfort from occurring due to the steam and sweat.

In an embodiment of the present invention, a binding layer is further included, which has a first binding face and a second binding face in opposite to the first binding face. The first binding face is removably combined with the release layer. The second binding face is adhered with a second combining face of the heating layer. Therefore, with the binding layer adhered to the clothing and the dressing layer adhered to the user skin, the heat patch of the present invention is prevented from accidentally detached, facilitating the adherence effect.

### BRIEF DESCRIPTION OF THE DRAWINGS

FIG. 1 is a perspective view of a first embodiment of the present invention;

FIG. 2 is an exploded view of the first embodiment of the present invention;

FIG. 3 is a sectional view of the first embodiment of the present invention;

FIG. 4 is a schematic view illustrating the heat patch in accordance with an embodiment of the present invention adhered to the user skin, wherein the heating layer is self-heating;

FIG. 5 is a schematic view illustrating the dressing layer adhered to the user skin;

FIG. 6 is a perspective view of a second embodiment of the present invention, wherein the dressing layer has a plurality of magnetic stones;

FIG. 7 is a perspective view of a third embodiment of the present invention, wherein the dressing layer has a plurality of bores;

FIG. 8 is a top view of the third embodiment of the present invention;

FIG. 9 is a perspective view of a fourth embodiment of the present invention, wherein the dressing layer has a plurality of concave grooves;

FIG. 10 is a top view of the fourth embodiment of the present invention;

FIG. 11 is a fifth embodiment of the present invention, wherein the heating layer is combined with the binding layer;

FIG. 12 is a perspective view of a sixth embodiment of the present invention;

FIG. 13 is a schematic view illustrating the binding layer adhered to the user skin.

### DETAILED DESCRIPTION

The aforementioned and further advantages and features of the present invention will be understood by reference to the description of the preferred embodiment in conjunction with the accompanying drawings where the components are illustrated based on a proportion for explanation but not subject to the actual component proportion.

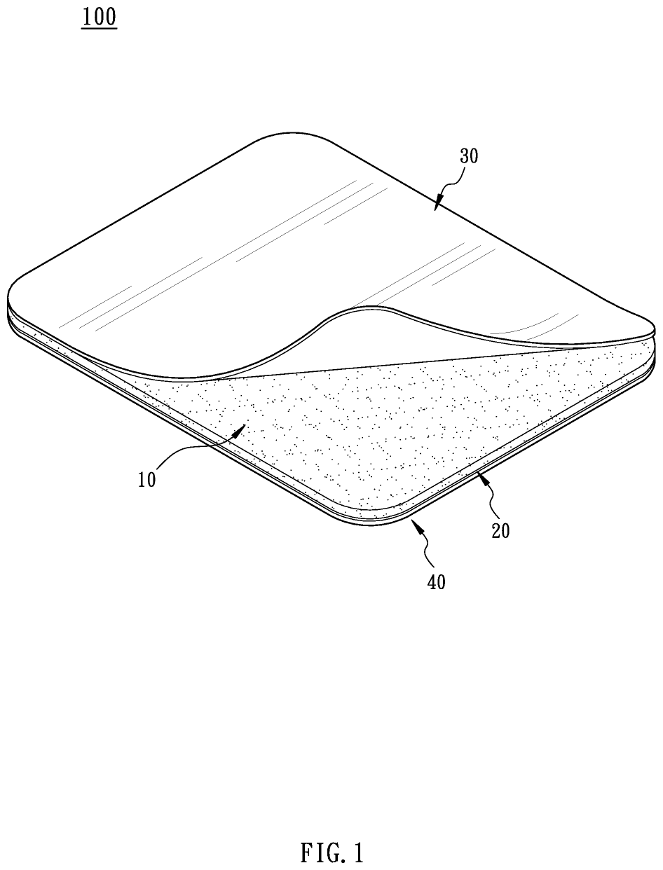

Referring to FIG. 1 to FIG. 3, a heat patch 100 in accordance with an embodiment of the present invention comprises a dressing layer 10, an adherence layer 20, a release layer 30, and a heating layer 40.

The dressing layer 10 has a first contact face 11 and a second contact face 12 in opposite to the first contact face 11. The first contact face 11 and the second contact face 12 are viscous faces, wherein the first contact face 11 has a first viscosity force, and the second contact face 12 has a second viscosity force. In an embodiment of the present invention, the first viscosity force is identical to the second viscosity force. Also, the dressing layer 10 includes an essential oil content and a Chinese herbal medicine content. The essential oil content is selected from essential oil made from lavender, sage, petitgrain, evening primrose, apricot kernel, mint, bergamot, ginger, and fennel seed, or other essential oil having equivalent effect. The Chinese medicine content is extracted by use of sesame oil as a base for acquiring the pharmaceutical effect of the Chinese medicine, with yellow wax or resin as an excipient or hardener thereof. Zinc oxide provides an antipruritic effect. Menthol, wintergreen oil, and borneol provides an anti-inflammatory, pain relieving, and cooling effects. The essential oil and Chinese herbal medicine content of the dressing layer 10 are allowed to be adjusted according to different symptoms to be relieved, therefore is not limited in the disclosure of the present invention.

The adherence layer 20 has a release face 21 and an adherence face 22, wherein the release face 21 is formed of a smooth material, such that the first contact face 11 of the dressing layer 10 is removably combined with the release face 21. The adherence face 22 is viscous and has a third viscosity force. The third viscosity force is larger than the first and second viscosity forces.

The release layer 30 is removably combined with the second contact face 12 of the dressing layer 10, wherein the release layer 30 is formed of a smooth material.

The heating layer 40 has a first combining face 41 and a second combining face 42, wherein the first combining face 41 and the second combining face 42 are formed of a non-woven fabric material. The first combining face 41 of the heating layer 40 is combined with the adherence face 22 of the adherence layer 20. In an embodiment of the present invention, the first combining face 41 is adhered with the adherence face 22.

Further, the heating layer 40 has a heating structure 43 disposed between the first combining face 41 and the second combining face 42. The heating structure 43 generates heat itself. In an embodiment of the present invention, the heating structure 43 is formed of a material including iron oxide, active carbon, salt, water and natural iron powder. When the heating structure 43 contacts the air, the heating structure 43 heats itself, such that the heat generated is spread through the non-woven fabric material of the first combining face 41 and the second combining face 42.

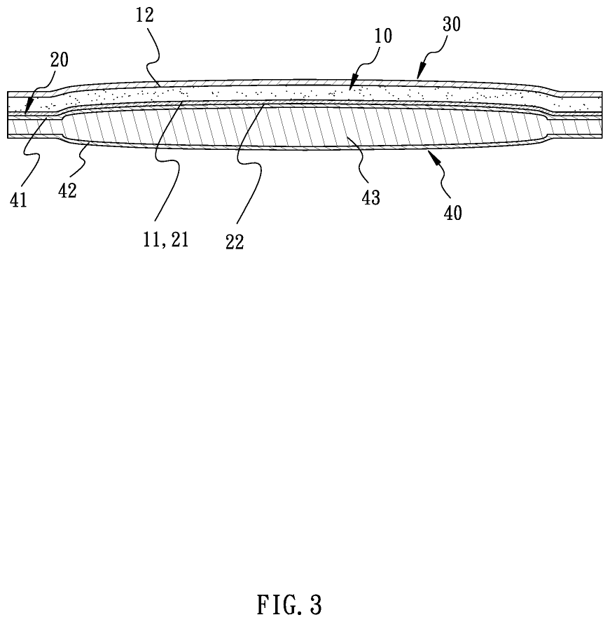

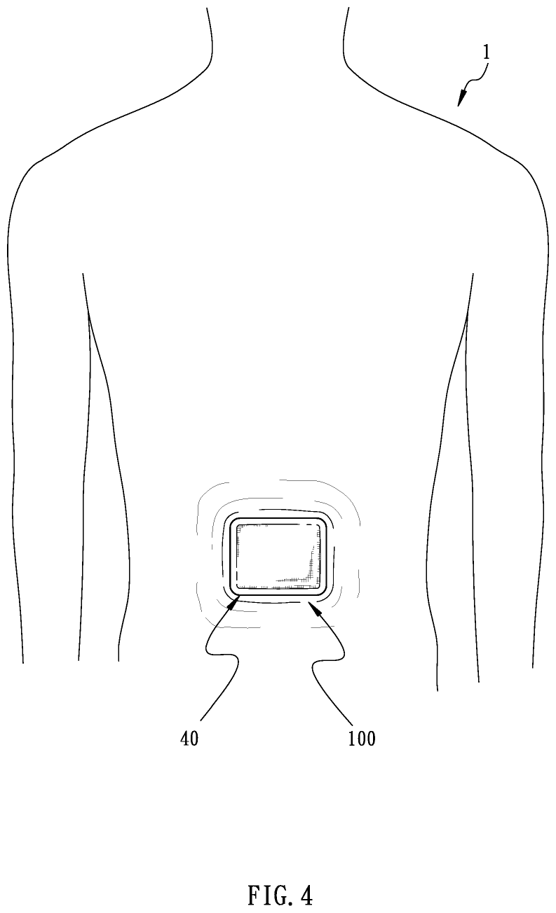

Referring to FIG. 3 to FIG. 4, when the user 1 applies the heat patch 100, the release layer 30 is removed from the second contact face 12 of the dressing layer 10, and the second contact face 12 of the dressing layer 10 is directly adhered to the skin of the user 1, such that the heating structure 43 of the heating layer 40 generates heat by itself. The heat thus generated is spread by the first combining face 41 and the second combining face 42, and transmitted to the dressing layer 10 through the adherence layer 20, facilitating the hot compressing function. Therefore, with the heating layer 40 combined with the dressing layer 10, the hot compressing function is provided for relieving discomfort of the user 1.

Also, the adherence layer 20 is disposed between the heating layer 40 and the dressing layer 10, so as to increase the combination between the heating layer 40 and the dressing layer 10, thus effectively preventing the heating layer 40 from being accidentally detached from the dressing layer 10.

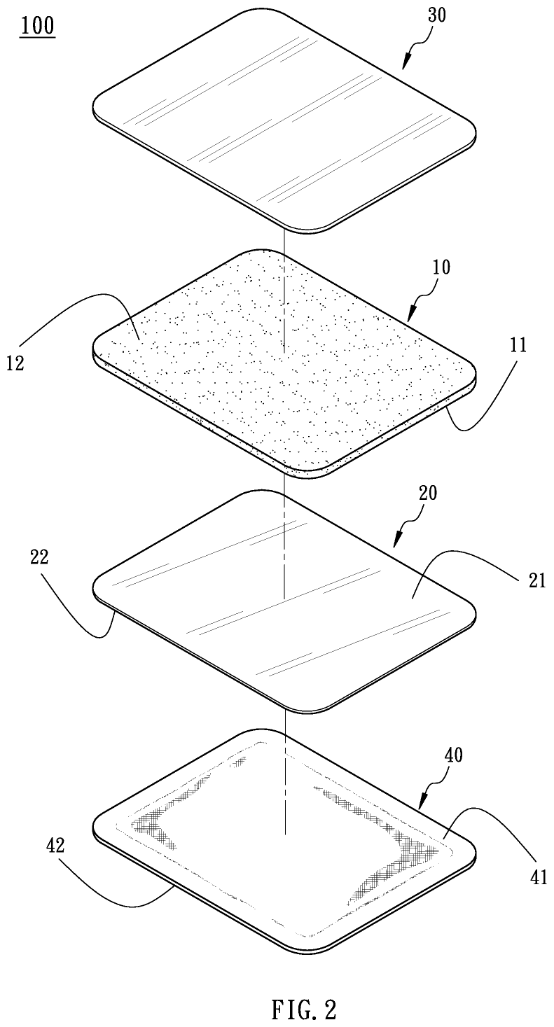

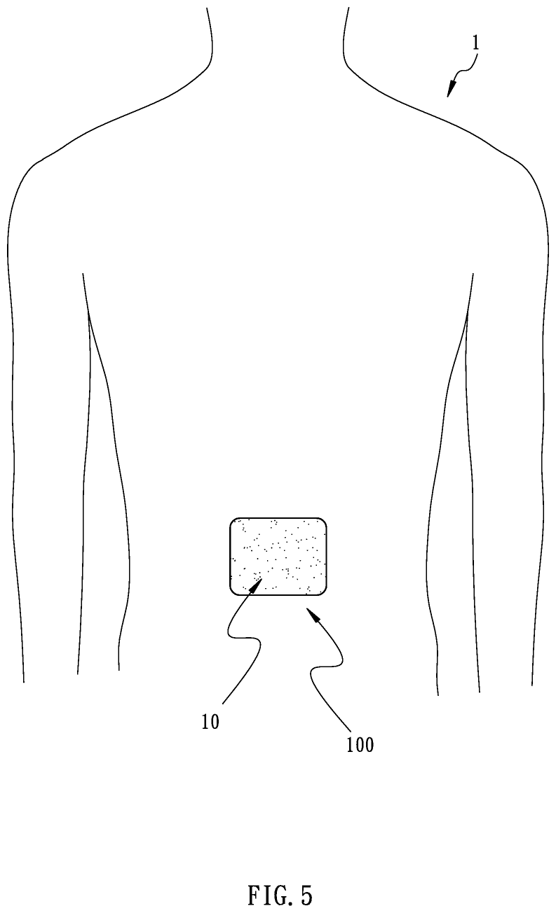

Referring to FIG. 2 and FIG. 5, the first contact face 11 and the second contact face 12 are both viscous. Therefore, the user 1 is able to remove the heating layer 40 and the adherence layer 20, and adhere the dressing layer 10 upon the skin of the user 1 for relieving the soreness.

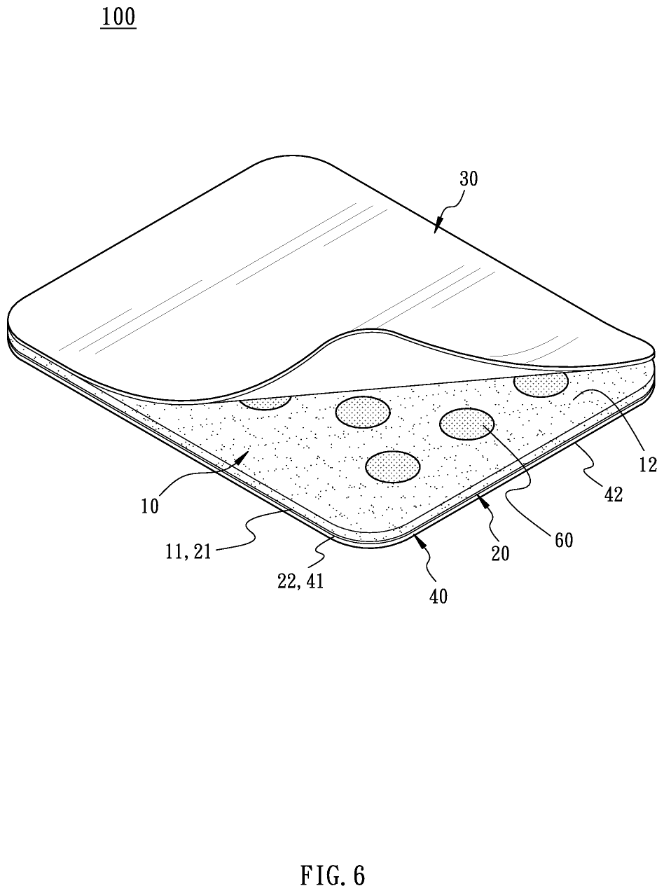

Referring to FIG. 6, the second embodiment of the heat patch 100 is illustrated, wherein numeric of similar or identical components are omitted.

In the second embodiment of the present invention, the second contact face 12 of the dressing layer 10 have a plurality of magnetic stones 60. Each magnetic stone 60 has magnetism. The magnetic stones 60 are allowed to be spaced with each other or scattered to be distributed on the second contact face 12. In an embodiment of the present invention, the magnetic stones 60 are distributed on the second contact face 12 of the dressing layer 10 in a scattering arrangement.

Referring to FIG. 4 and FIG. 6, when the second contact face 12 of the dressing layer 10 is adhered to the skin of the user 1, the magnetic stones 60 applied with the essential oil or Chinese medicine content of the dressing layer 10 are able to enhance the pain relieving effect.

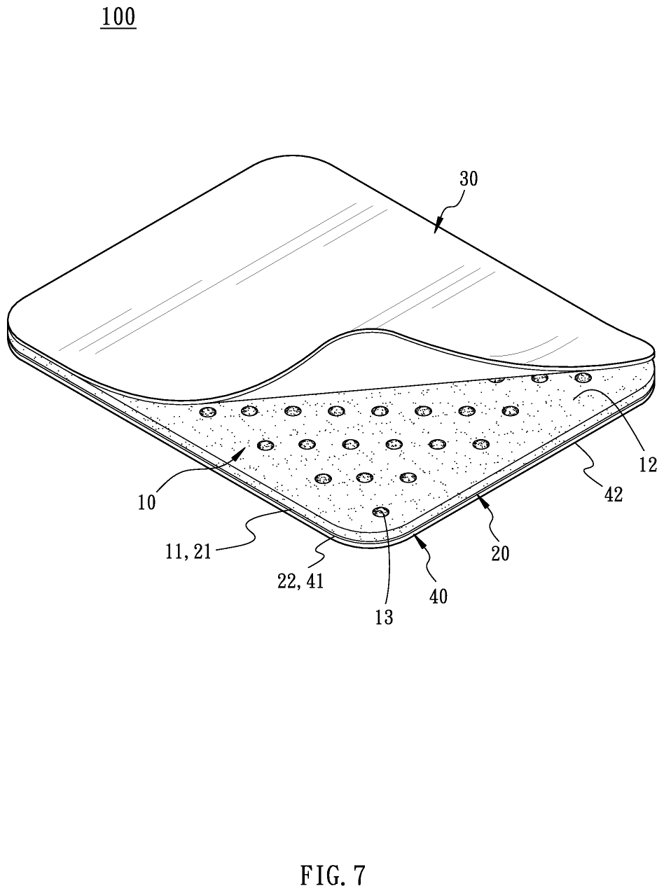

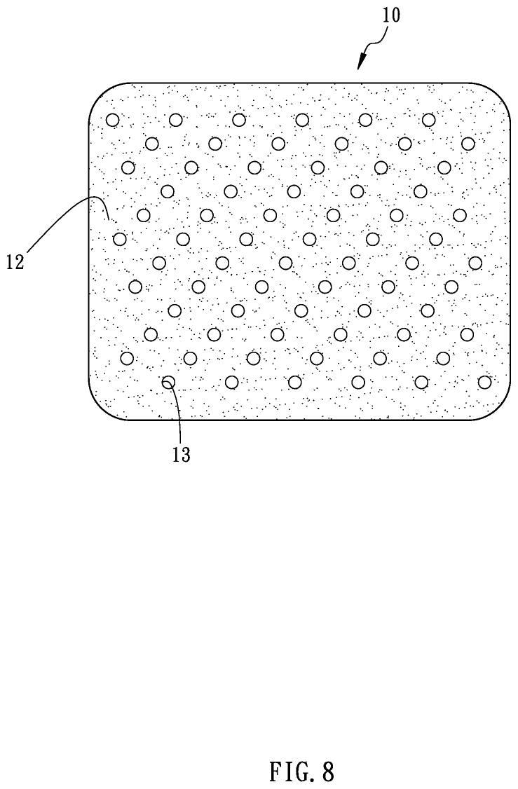

Referring to FIG. 7 to FIG. 8, the third embodiment of the heat patch 100 is illustrated, wherein numeric of similar or identical components are omitted.

In the third embodiment of the present invention, a plurality of bores 13 pass through the dressing layer 10, wherein the bores 13 are equidistantly distributed on the dressing layer 10.

Referring to FIG. 3 to FIG. 4 and FIG. 7 to FIG. 8, when the heat generated by the heating structure 43 of the heating layer 40 is transmitted to the skin of the user 1 through dressing layer 10, the steam and sweat, which are possibly produced by the skin of the user 1 are discharged through the bores 13, thus preventing the discomfort due to the steam and sweat from occurring upon the user 1. Therefore, the permeability of the dressing layer 10 is improved.

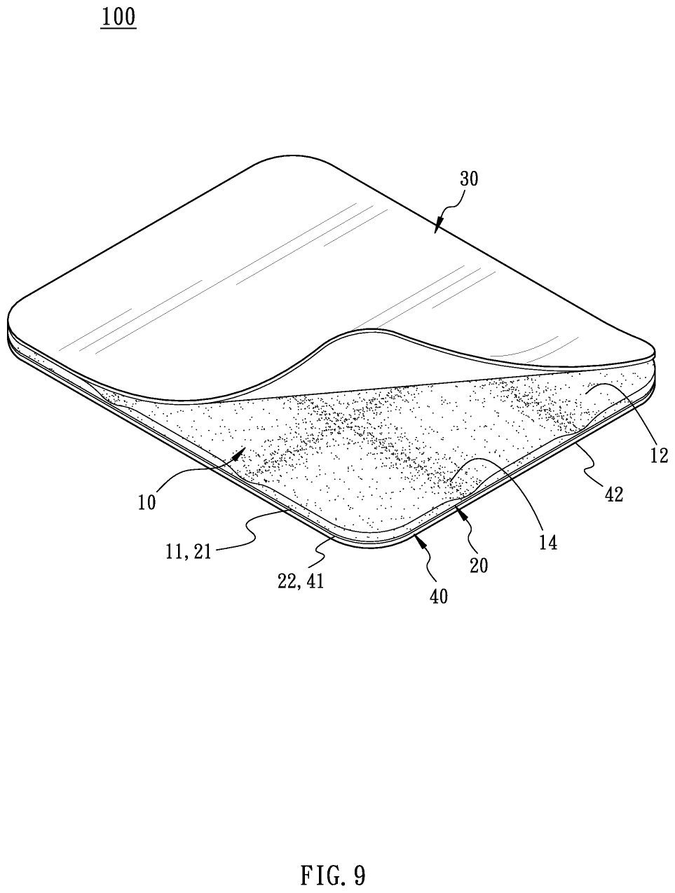

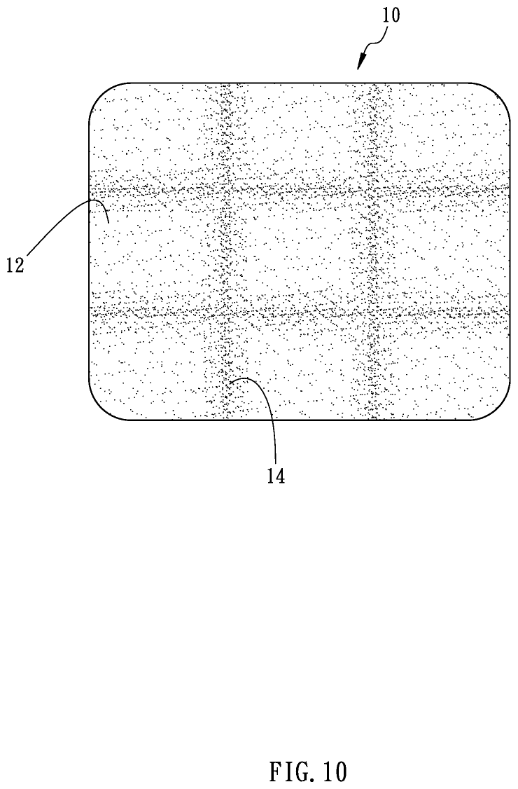

Referring to FIG. 9 to FIG. 10, the fourth embodiment of the heat patch 100 is illustrated, wherein numeric of similar or identical components are omitted.

In the fourth embodiment of the present invention, the second contact face 12 of the dressing layer 10 have a plurality of grooves 14, wherein the grooves 14 are intercrossed. In the embodiment, the grooves 14 are connected at the intersection thereof.

Referring to FIG. 4 and FIG. 9 to FIG. 10, when the heat generated by the heating structure 43 is transmitted to the skin of the user 1 through the dressing layer 10, the steam and sweat, which are possibly produced by the skin of the user 1 are discharged away from the skin through the grooves 14, thus preventing the discomfort due to the steam and sweat from occurring upon the user 1. Therefore, the permeability of the dressing layer 10 is improved.

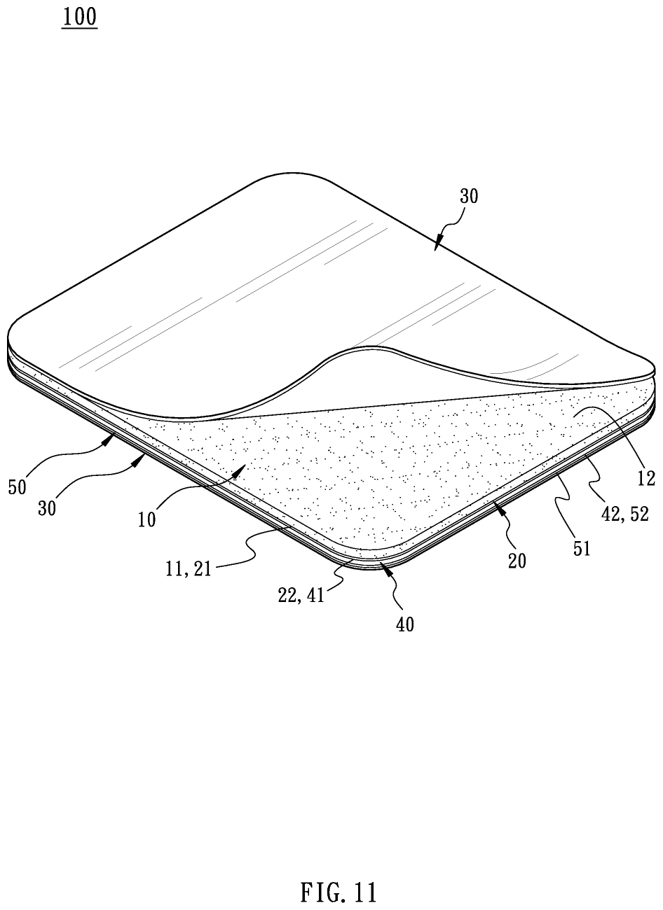

Referring to FIG. 11, the fifth embodiment of the heat patch 100 is illustrated, wherein numeric of similar or identical components are omitted. In the fifth embodiment, the heat patch 100 further comprises a binding layer 50.

The binding layer 50 is combined with the release layer 30 and the heating layer 40. The binding layer 50 has a first binding face 51 which is removable combined with the release layer 30.

In the embodiment, the binding layer 50 further has a second binding face 52 in opposite to the first binding face 51 and combined with the second combining face 42. Therefore, two release layers 30 are included, with one release layer 30 adhered to the second contact face 12 of the dressing layer 10 and the other release layer 30 adhered to the first binding face 51 of the binding layer 50.

When the two release layers 30 are removed from the second contact face 12 of the dressing layer 10 and the first binding face 51 of the binding layer 50, respectively, the first binding face 51 of the binding layer 50 is able to be adhered to the clothe, and the second contact face 12 of the dressing layer 10 is able to be adhered to the skin of the user 1. Therefore, the heat patch 100 of the present invention is effectively positioned to the soreness-relieving needed portion of the skin of the user 1, such that the binding layer 50 effectively prevents the heat patch 100 from accidental detachment.

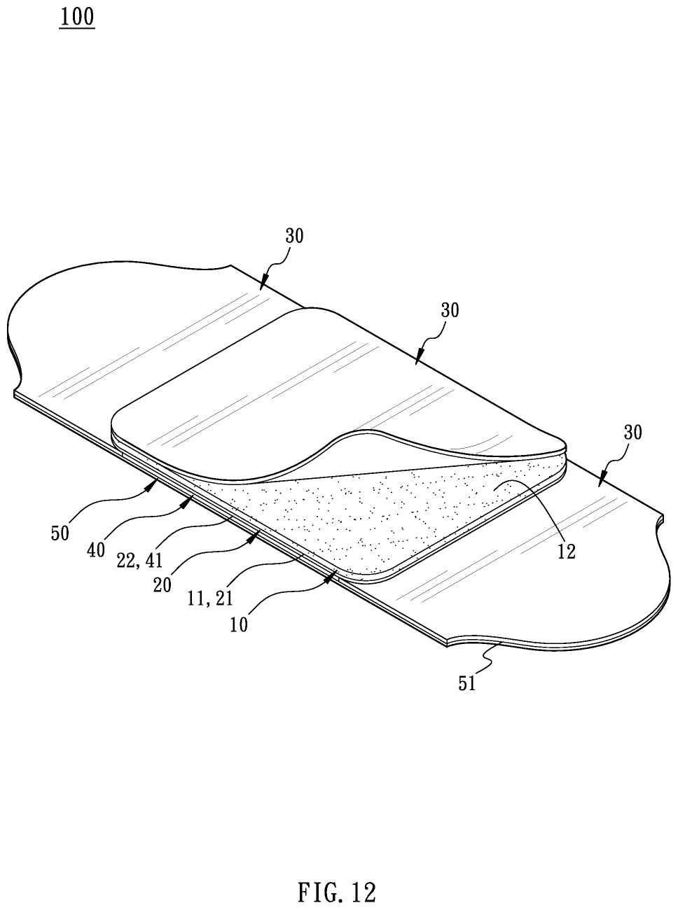

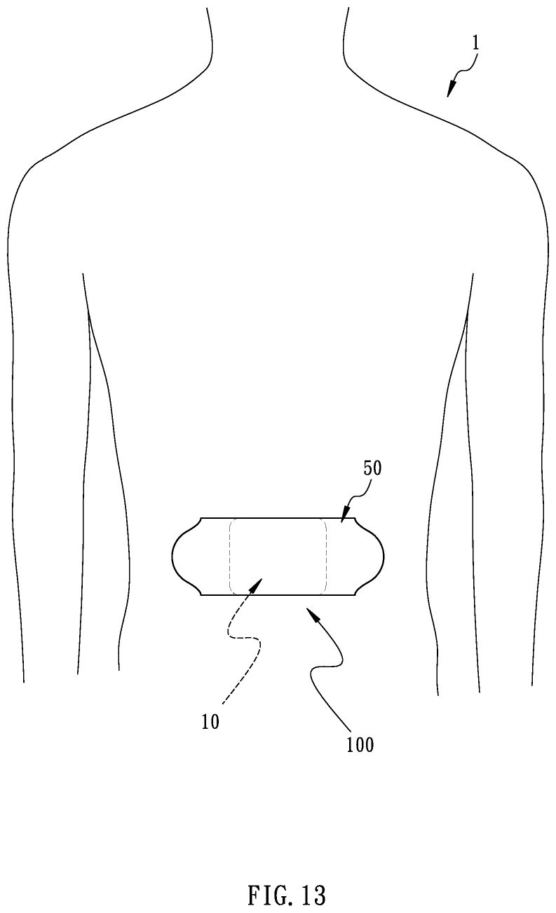

Referring to FIG. 12 and FIG. 13, the sixth embodiment of the heat patch 100 is illustrated, wherein numeric of similar or identical components are omitted.

In the sixth embodiment, the area of the binding layer 50 is larger than the area of the dressing layer 10, the adherence layer 20, and the heating layer 40. In the embodiment, the dressing layer 10, the adherence layer 20, and the heating layer 40 are arranged at the center of the binding layer 50.

The first binding face 51 of the binding layer 50 is combined with the second combining face 42 of the heating layer 40 and the release layer 30. Therein, two release layers 30 are included, with one release layer 30 combined to the second contact face 12 of the dressing layer 10, and the other release layer 30 combined to the first binding face 51 of the binding layer 50.

When the two release layers 30 are removed from the second contact face 12 of the dressing layer 10 and the first binding face 51 of the binding layer 50, respectively, the first binding face 51 of the binding layer 50 and the second contact face 12 of the dressing layer 10 are able to be adhered to the skin of the user 1. Therefore, with the first binding face 51 having a larger area, the heat patch 100 of the present invention is effectively positioned to the soreness-relieving needed portion of the skin of the user 1, effectively preventing the heat patch 100 from accidental detachment.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A heat patch, comprising:
a dressing layer having a first contact face and a second contact face in opposite to the first contact face;
a release layer removably disposed on the second contact face of the dressing layer;
an adherence layer having a release face and an adherence face in opposite to the release face, the first contact face of the dressing layer removably disposed on the release face; and
a heating layer having one lateral side disposed on the adherence face, the heating layer further having a heating structure.

2. The heat patch of claim 1, wherein the heating layer has a first combining face and a second combining face in opposite to the first combining face, and the heating structure is disposed between the first combining face and the second combining face.

3. The heat patch of claim 2, wherein the adherence face is viscous and adhered to the first combining face.

4. The heat patch of claim 1, wherein the first contact face and the second contact face of the dressing layer are viscous.

5. The heat patch of claim 4, wherein the first contact has a first viscosity force, the second contact face has a second viscosity force, and the adherence face has a third viscosity force; the first viscosity force is identical to the second viscosity force, and the third viscosity force is larger than the first viscosity force and the second viscosity force.

6. The heat patch of claim 2, wherein the first combining face and the second combining face are formed of a non-woven fabric material.

7. The heat patch of claim 1, wherein the second contact face of the dressing layer have a plurality of magnetic stones.

8. The heat patch of claim 1, wherein a plurality of bores pass through the dressing layer and are spaced with each other.

9. The heat patch of claim 1, wherein the second contact face of the dressing layer has a plurality of concave grooves.

10. The heat patch of claim 9, wherein the grooves are intercrossed.

11. The heat patch of claim 1, further comprising a binding layer combined with the release layer and the heating layer, the binding layer having a first binding face, the first binding face removably combined with the release layer.

12. The heat patch of claim 11, wherein the binding layer has a second binding face disposed in opposite to the first binding face, and the second binding face is combined with the heating layer.

13. The heat patch of claim 11, wherein the binding layer has an area which is larger than areas of the dressing layer, the binding layer, and the heating layer, and the heating layer is combined with the first binding face.

14. The heat patch of claim 1, wherein the dressing layer has an essential oil content and a Chinese medicine content.
